(19) United States
(12) Patent Application Publication
(10) Pub. No.: US 2025/0139764 A1
(43) Pub. Date: May 1, 2025

(54) SYSTEM AND METHOD FOR MOTION GUIDED RETROSPECTIVE GATING

(71) Applicant: GE Precision Healthcare LLC, Waukesha, WI (US)

(72) Inventors: Arka Datta, Pewaukee, WI (US); Brian E. Nett, Wauwatosa, WI (US); Chelsey Amanda Lewis, Waukesha, WI (US)

(21) Appl. No.: 18/499,071

(22) Filed: Oct. 31, 2023

(51) Int. Cl. G06T 7/00; G06T 7/246; G06T 11/00

(52) U.S. Cl. CPC G06T 7/0012; G06T 7/248; G06T 11/005; G06T 11/008; G06T 2207/10028; G06T 2207/10081; G06T 2207/10116; G06T 2207/30004; G06T 2210/41; G06T 2211/428; G06T 2211/464

(57) ABSTRACT

An imaging system and method for using the imaging system are described. In one example, a method of A method of identifying movement of a patient during a medical imaging scan includes initiating motion detection data acquisition, initiating a medical imaging scan of the patient to acquire scan data, determining a motion score curve for duration of the medical imaging scan based on the motion detection data, removing portions of the scan data corresponding to a motion score curve outside of an acceptable range, and selecting the scan data corresponding to a motion score curve within the acceptable range for reconstruction.

### BACKGROUND

The subject matter disclosed herein relates to medical imaging systems and, more particularly, to detecting motion during a computed tomography (CT) scan using one or more sensors or systems to detect the motion. The sensors may include cameras and/or laser imaging, detection and ranging (LiDAR) based techniques incorporated within a CT imaging system.

In CT, X-ray radiation spans a subject of interest, such as a human patient, and a portion of the radiation impacts a detector where the image data is collected. In digital X-ray systems a photodetector produces signals representative of the amount or intensity of radiation impacting discrete pixel regions of a detector surface. The signals may then be processed to generate an image that may be displayed for review. In the images produced by such systems, it may be possible to identify and examine the internal structures and organs within a patient's body. In CT imaging systems a detector array, including a series of detector elements or sensors, produces similar signals through various positions as a gantry is rotated around a patient, allowing volumetric reconstructions to be obtained.

An accurate three-dimensional (3D) measurement of a patient during a CT can significantly improve subsequent workflow (e.g., patient positioning, automated landmarking, etc.). For example, using the 3D measurement and of a patient and position information obtained from the measurement can help determine movement of the patient.

### SUMMARY

Certain embodiments commensurate in scope with the originally claimed subject matter are summarized below. These embodiments are not intended to limit the scope of the claimed subject matter, but rather these embodiments are intended only to provide a brief summary of possible forms of the subject matter. Indeed, the subject matter may encompass a variety of forms that may be similar to or different from the embodiments set forth below. This summary introduces concepts that are described in more detail in the detailed description. It should not be used to identify essential features of the claimed subject matter, nor to limit the scope of the claimed subject matter.

In one aspect, a method of identifying movement of a patient during a medical imaging scan. The method includes initiating motion detection data acquisition, initiating a medical imaging scan of the patient to acquire scan data, determining a motion score curve for duration of the medical imaging scan based on the motion detection data, removing portions of the scan data corresponding to a motion score curve outside of an acceptable range, and selecting the scan data corresponding to a motion score curve within the acceptable range for reconstruction.

In another aspect, a medical imaging system is provided. The medical imaging system includes a computed tomography (CT) imaging system, which includes a gantry having a bore, rotatable about an axis of rotation, an X-ray source mounted on the gantry and configured to emit an X-ray beam, an X-ray controller to operate the X-ray source, and an X-ray detector configured to detect the X-ray beam emitted by the X-ray source. The medical imaging system also includes a motion detection system coupled to the CT imaging system, which includes a motion detection apparatus mounted on the gantry, a controller to control operation of the motion detection apparatus, and a movement data processing unit to obtain data from the motion detection apparatus, wherein the movement data processing unit obtains a baseline position of a patient and real-time movement data during an imaging scan to generate additional position information, wherein the real-time movement data is compared to the baseline position of the patient. The system also includes a processor to determine a motion score of the patient for each view during the scan and select views of scan data in which the corresponding motion score is in an acceptable range for image reconstruction.

### BRIEF DESCRIPTION OF THE DRAWINGS

These and other features, aspects, and advantages of the present disclosed subject matter will become better understood when the following detailed description is read with reference to the accompanying drawings in which like characters represent like parts throughout the drawings, wherein:

FIG. 1 is a pictorial representation of a CT imaging system, in accordance with aspects of the present disclosure;

FIG. 2 is a block diagram of the CT imaging system in FIG. 1, in accordance with aspects of the present disclosure;

FIG. 3 is a schematic diagram of a motion detection system window placement within a gantry of a CT imaging system, in accordance with aspects of the present disclosure;

FIG. 4 is a perspective view of a schematic diagram of a motion detection system relative to a patient on a CT table, in accordance with aspects of the present disclosure;

FIGS. 5A-5C depict a single motion detection apparatus obtaining data at different angular positions, in accordance with aspects of the present disclosure;

FIG. 6 is a pictorial representation of a single motion detection apparatus rotating 360 degrees around a patient for data acquisition, in accordance with aspects of the present disclosure;

FIG. 7 is a pictorial representation of multiple motion detection apparatuses rotating around a patient for data acquisition, in accordance with aspects of the present disclosure;

FIG. 8 is a perspective view of a schematic diagram of multiple motion detection apparatuses obtaining data of a patient at different angular positions at the same time; in accordance with aspects of the present disclosure;

FIG. 9 is a pictorial representation of motion detection apparatuses placed 360 degrees around the patient for data acquisition, in accordance with aspects of the present disclosure;

FIG. 10 is a pictorial representation of an external motion detection system coupled to a CT table, in accordance with aspects of the present disclosure;

FIG. 11 is a flowchart of a method for motion guided retrospective gating in a CT imaging system;

FIG. 12 is a flowchart of a method of processing patient movement data for use in conjunction with the method of FIG. 11;

FIG. 13 is a flowchart of a method of performing retrospective gating for use in conjunction with the method of FIG. 11;

FIG. 14 is an illustrated example of a selected surface area of interested to be monitored for motion during a scan;

FIG. 15 depicts an example range of motion that is acceptable during a scan;

FIGS. 16A-16C depict example data set depicting acceptable and unacceptable motion during a scan; and

FIGS. 17A-17E depict an example of scan data that may be selected based on the retrospective gating.

### DETAILED DESCRIPTION

Embodiments of the present disclosure will now be described, by way of example, with reference to the Figures. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

While aspects of the following discussion may be provided in the context of medical imaging, it should be appreciated that the present techniques are not limited to such medical contexts. Indeed, the provision of examples and explanations in such a medical context is only to facilitate explanation by providing instances of real-world implementations and applications. However, the present approaches may also be utilized in other contexts, such as tomographic image reconstruction for industrial Computed Tomography (CT) used in non-destructive inspection of manufactured parts or goods (i.e., quality control or quality review applications), and/or the non-invasive inspection of packages, boxes, luggage, and so forth (i.e., security or screening applications). In general, the present approaches may be useful in any imaging or screening context to provide accurate three-dimensional (3D) information of a target to improving workflow processes and post processing steps.

The present disclosure provides systems and methods for monitoring patient motion during a CT scan, which may include incorporating laser imaging, detection and ranging (LiDAR) based techniques, motion sensors, and/or 3D cameras with a CT imaging system. An example motion detection system includes a LiDAR system, which is a remote sensing method to measure target objects or patients that a variable distance from an X-ray source. With the advancement of LiDAR technique, it can now produce 3D rendering of a subject with high spatial resolution (e.g., sub millimeter (mm) accuracy). The disclosed techniques of motion detection using a LiDAR technique use time of flight information of reflected pulsed light (e.g., laser) to calculate and reproduce 3D information (e.g., depth dependent information) of the patient. The time of flight information may calculate depth, which may be used to create a 3D model of a patient undergoing a CT scan and to detect motion of the patient undergoing the CT scan. Multiple views are utilized to cover an entire target area to reproduce high fidelity 3D information. In certain embodiments, light images may be acquired by moving the data acquisition system (i.e., LiDAR scanning system having one or more LiDAR scanners or instruments) around the target (e.g., around the gantry). Using these techniques, a LiDAR-based motion detection system is able to detect motion of a patient in real-time with accuracy. Additionally or alternatively, other types of motion sensors and/or 3D cameras may be used to track the motion of the patient. For example, a motion detection system using a 3D camera (e.g., a depth camera) may create a 3D mesh that can be updated in real time to monitor the movement of the patient.

In some embodiments, the motion detection system is integrated within the CT imaging system. In this embodiment, the data acquisition system may be integrated outside the scan window (and, thus, physically coupled to the CT imaging system) and rotated to capture multiple views. In certain embodiments, multiple motion detection apparatuses or instruments may be placed at different angular positions around the patient to capture the entire region of interest. In certain embodiments, a motion detection system (e.g., having multiple motion detection apparatuses or instruments) may be mounted externally relative to the gantry (e.g., on the scanner housing or the CT table) but still be physically coupled to the CT imaging system. The external motion detection system may be placed in position as required by a guided rail system. The patient movement data may be acquired prior to, during, and subsequent to a CT scan of the target or patient. The patient movement data can be processed and utilized for subsequent (i.e., after the LiDAR scan) workflow processes (e.g., accurate light scout measurement, proper patient positioning and automated landmarking, etc.) and post-processing steps (e.g., image reconstruction). The disclosed embodiments provide a holistic framework for including a motion detection system in a CT imaging system to improve overall efficiency and robustness of the workflow processes and post-processing steps.

The present disclosure also provides a method of detecting a position and movement of an object or patient positioned for an imaging scan using the motion detection system. The example motion detection system provides data related to the position and movement of the object, specifically 3D point cloud or 3D mesh information of the object or patient. The position and movement information data can be compared to an imaging protocol to determine if the position or movement of the object corresponds to the protocol (i.e., the right portion of the object is positioned to be imaged, the movement of the patient is within an acceptable range to provide quality scan data). If movement outside an acceptable range is detected, the imaging system can account for the movement by removing the view from the imaging scan that include movement. Specifically, the imaging system can select a continuous portion of the data that has only movement in an acceptable range for use during reconstruction, and discard data containing movement falling outside of the acceptable range. Thus, the image quality can be improved by accounting for the movement of the subject.

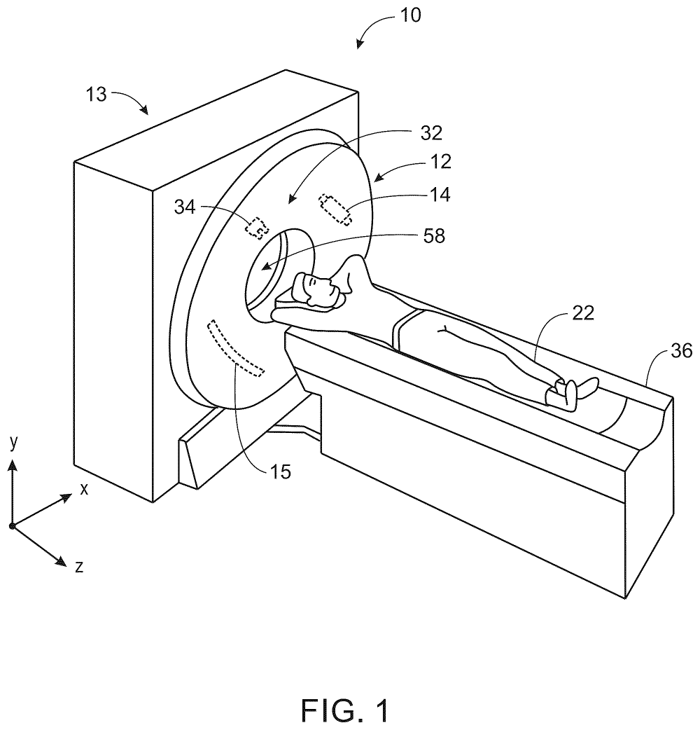

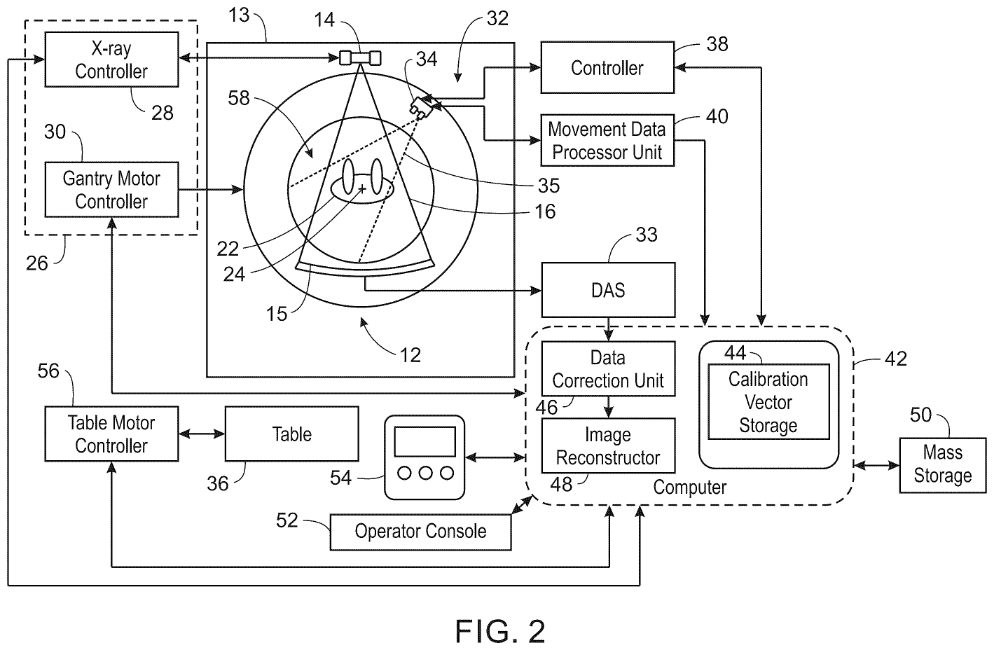

With the preceding in mind and referring to FIGS. 1 and 2, a CT imaging system 10 is shown, by way of example. The CT imaging system 10 includes a gantry 12 enclosed within a housing 13 (e.g., gantry housing). The gantry 12 has a rotating component and a stationary component. The gantry 12 has an X-ray source 14 that projects a beam of X-rays 16 toward an X-ray detector assembly or X-ray detector array 15 (e.g., having a plurality of detector modules) on the opposite side of the gantry 12. The X-ray source 14 and the X-ray detector assembly 15 are disposed on the rotating portion of the gantry 12. The X-ray detector assembly 15 is coupled to a data acquisition system (DAS) 33. The plurality of detector modules of the X-ray detector assembly 15 detect the projected X-rays that pass through a patient or subject 22, and DAS 33 converts the data to digital signals for subsequent processing. Each detector module of the X-ray detector assembly 15 in a conventional system produces an analog electrical signal that represents the intensity of an incident X-ray beam and hence the attenuated X-ray beam as it passes through the patient 22. During a scan to acquire X-ray projection data, gantry 12 and the components mounted thereon rotate about a center of rotation 24 (e.g., isocenter) so as to collect attenuation data from a multitude of view angles relative to the imaged volume.

Rotation of gantry 12 and the operation of X-ray source 14 are governed by a control mechanism 26 of CT imaging system 10. Control mechanism 26 includes an X-ray controller 28 that provides power and timing signals to the X-ray source 14 and a gantry motor controller 30 that controls the rotational speed and position of gantry 12.

The imaging system 10 also includes motion detection system 32 physically coupled to the imaging system 10. In some examples, the motion detection system includes a LiDAR system and/or 3D camera(s). The motion detection system 32 includes one or more motion detection apparatuses or instruments 34. As depicted, the motion detection system 32 has one motion detection apparatus 34. The one or more motion detection apparatuses 34 are utilized to acquire depth dependent information (e.g., LiDAR data or light images, 3D mesh data) of the patient 22 with high spatial fidelity. The depth dependent information is utilized in subsequent workflow processes for a CT scan. In examples where LiDAR techniques are used, the one or more motion detection apparatuses 34 emit pulsed light 35 (e.g., laser) at the patient 22 and detect the reflected pulsed light from the patient 22. The motion detection system 32 is configured to acquire the movement data from multiple different views (e.g., at different angular positions relative to the axis of rotation 24).

In certain embodiments, as depicted in FIGS. 1 and 2, the motion detection apparatus 34 is coupled to the gantry 12. In particular, the motion detection apparatus 34 is disposed within the gantry housing 13 outside a scan window. The motion detection apparatus 34 is rotated around the patient 22 to acquire the movement data at the different angular positions. In certain embodiments, multiple motion detection apparatuses 34 may be coupled to the gantry 12 and rotated to acquire the movement data at the different angular positions.

In certain embodiments, multiple motion detection apparatuses 34 may be coupled to the gantry 12 in fixed positions but disposed at different angular positions (e.g., relative to axis of rotation 24). In the illustrated example, the motion detection apparatuses 34 are depicted as LiDAR scanners, but other suitable means to detect motion (e.g., 3D cameras, sensors, etc.) may be used in addition and/or in place of the LiDAR scanners. The motion detection apparatuses 34 in fixed positions may acquire the movement data at the same time while remaining stationary.

In certain embodiments, the motion detection system 32 may be external to the gantry 12 but still physically coupled to the imaging system 10. For example, multiple motion detection apparatuses 34 may be coupled to a panel (e.g., at different angular positions relative to the axis of rotation 24) that is coupled to a guide rail system. The guide rail system may be coupled to the gantry housing 13 or a table 36 of the system 10. The guide rail system may be configured to move the panel toward and away from the gantry 12. In certain embodiments, the guide rail system may also be configured to rotate the panel about the axis of rotation 24.

The motion detection system 32 includes a controller 38 configured to provide timing and control signals to the one or more motion detection apparatuses 34 for acquiring the movement data at the different angular positions. The movement data may be acquired prior to, during, and/or subsequent to a CT scan of the patient 22. The motion detection system 32 also includes a movement data processing unit 40 that receives or obtains the movement data from the one or more motion detection apparatuses 34. The integrated motion detection system and/or the movement data processing unit 40 utilizes time of flight information of the reflected pulsed light and processes the movement data (e.g., acquired at the different views) to generate an accurate 3D measurement of the patient 22. The 3D measurement of the patient 22 has a high spatial resolution (e.g., sub mm accuracy). As noted above, the 3D measurement may be utilized in subsequent workflow processes of a CT scan. For example, the 3D measurement may be utilized as an accurate light scout measurement (e.g., for modifying scan acquisition parameters). The 3D measurement may also be utilized for proper patient positioning (e.g., for modifying or optimizing patient position parameters) and automated landmarking. The 3D measurement may further be utilized for post-processing such as in an image reconstruction algorithm of the CT scan data (e.g., modifying reconstruction parameters).

The 3D measurement information from the motion detection system 32 (e.g., from the movement data processing unit 40) and the scan data from the DAS 33 is input to a computer 42. The computer 42 includes a calibration vector storage 44 (e.g., for storing calibration parameters and calibration protocols for acquiring the CT scan data). The 3D measurement information obtained from the motion detection system 32 may be utilized in determining the calibration parameters utilized. The computer 42 also includes a data correction unit 46 for processing or correcting the CT scan data from the DAS 33. The computer 42 further includes an image reconstructor 48. The image reconstructor 48 receives sampled and digitized X-ray data from DAS 33 and performs high-speed reconstruction. The reconstructed image is applied as an input to the computer 42, which stores the image in a mass storage device 50. Computer 42 also receives commands and scanning parameters from an operator via console 52. An associated display 54 allows the operator to observe the reconstructed image as well as the 3D measurement data and other data from the computer 42. The operator supplied commands and parameters are used by computer 42 to provide control signals and information to the DAS 33, X-ray controller 28, gantry motor controller 30, and the controller 38. In addition, computer 42 operates a table motor controller 56, which controls a motorized table 36 to position the patient 22 relative to the gantry 12. Particularly, table 36 moves portions of the patient 22 through a gantry opening or bore 58.

The computer 42 and the movement data processing unit 40 include may each include processing circuitry. The processing circuitry may be one or more general or application-specific microprocessors. The processing circuitry may be configured to execute instructions stored in a memory to perform various actions. For example, the processing circuitry may be utilized for receiving or obtaining movement data acquired with the motion detection system 32. In addition, the processing circuitry may also generate a 3D measurement of the patient 22. Further, the processing circuitry may utilize the 3D measurement in a subsequent workflow process for a CT scan of the patient with the CT imaging system 32.

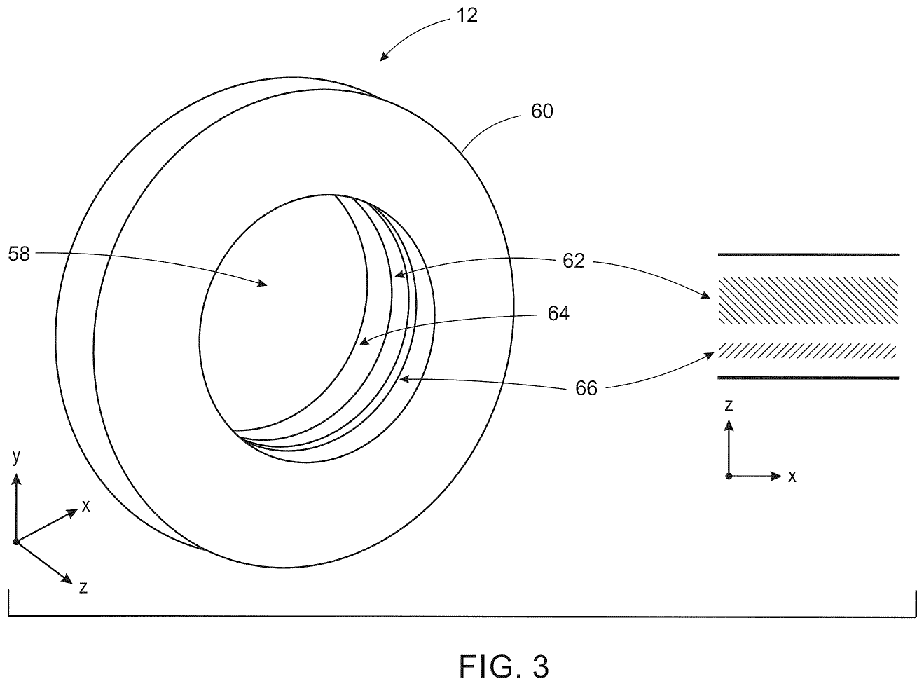

FIG. 3 is a schematic diagram of motion detection system window placement within the gantry 12 of a CT imaging system. The gantry 12 includes a gantry cover 60 configured to be located on a front portion of the gantry housing adjacent the CT table, and located on a rear portion of the gantry housing. The CT imaging system includes an annular scan window 62 disposed within an interior wall 64 of the gantry 12 formed within the opening or the bore 58 of the gantry 12. The scan window 62 is made of an X-ray transparent material that enables X-rays emitted from an X-ray source to pass through an object or subject being imaged for detection by a detector. The scan window 62 is constructed so as to be fitted between components of the housing of the gantry 12 (e.g., front and back covers) to fill a gap. During an imaging session, a subject or patient is moved within the bore 58. The scan window 62 self-supports and acts as a safety barrier to keep the subject or patient from contacting rotating components within the gantry 12.

As depicted, an annular motion detection system panel or window 66 is also disposed within the interior wall 64 of the gantry 12 formed within the bore 58 of the gantry 12. The panel 66 is made of a material transparent to the pulsed light (e.g., laser) emitted by one or more motion detection apparatus disposed within gantry 12 toward the object or subject and reflected back to the one or more motion detection apparatus. The panel 66 is disposed between scan window 62 and the gantry cover 60 in the Z-direction. In particular, the panel 66 is disposed between the scan window 62 and a front of the gantry 12 adjacent the CT table. During an imaging session, a subject or patient is moved within the bore 58. The panel 66 also self-supports and acts as a safety barrier to keep the subject or patient from contacting components (e.g., sometimes rotating components) within the gantry 12. The one or more motion detection apparatuses may be located within the gantry 12 behind the panel 66. In certain embodiments, the one or more motion detection apparatuses 34 are stationary during acquisition of movement data. In certain embodiments, the one or more motion detection apparatuses rotate during acquisition of movement data. The panel 66 is located outside the region of the subject or object being scanned by the CT imaging system.

FIG. 4 is a perspective view of a schematic diagram of the motion detection system 32 relative to the patient on the CT table 36. With the exception of the motion detection system 32, the components of the gantry are not shown for simplification. One or more motion detection apparatuses 34 may be coupled to the panel or window 66. As depicted, a single motion detection apparatus 34 is coupled to the panel 66. In certain embodiments, multiple motion detection apparatuses 34 may be disposed at different circumferential positions (e.g., different angular positions relative to the axis of rotation 24 in FIG. 2) along the panel 66. The panel 66 is disposed within the inner wall of the gantry formed within the bore of the gantry. The motion detection apparatuses 34 are arranged in a concentric manner with the panel 66 (e.g., relative to the axis of rotation 24) with the panel 66 being located radially closer (in the Y-direction) than the motion detection apparatuses 34 to the axis of rotation 24. In certain embodiments, the panel 66 and associated motion detection apparatuses 34 remain in a fixed position, while motion detection apparatuses 34 acquire the movement data at different angular positions. In certain embodiments, the panel 66 and one or more associated motion detection apparatuses 34 rotate to acquire the movement data.

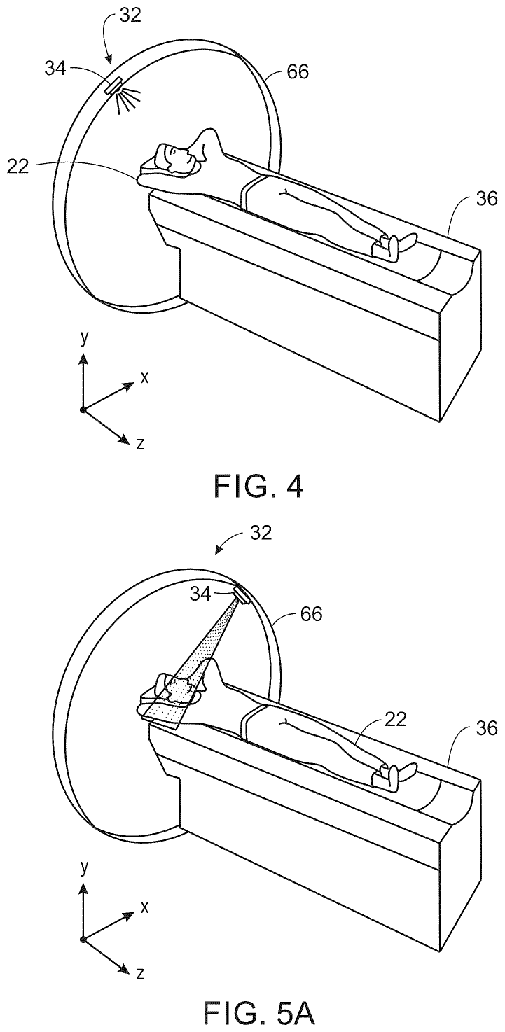

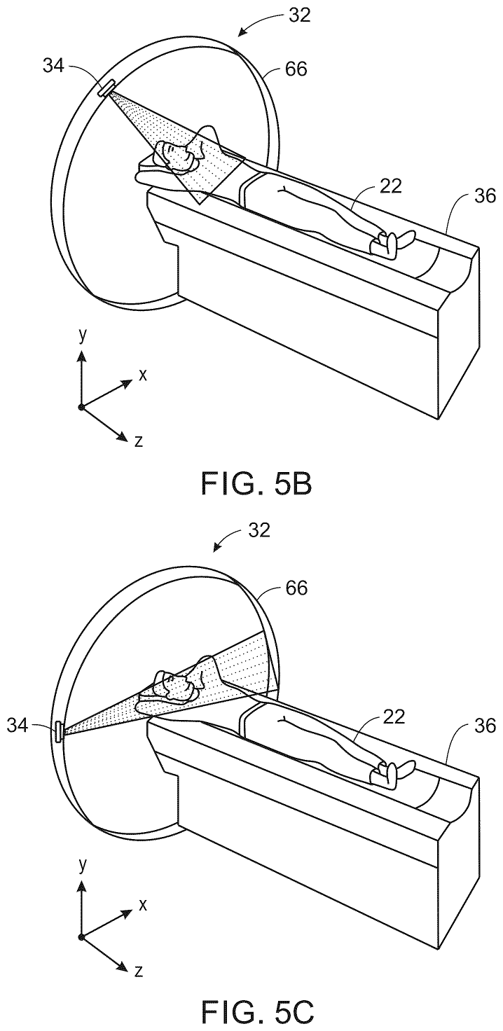

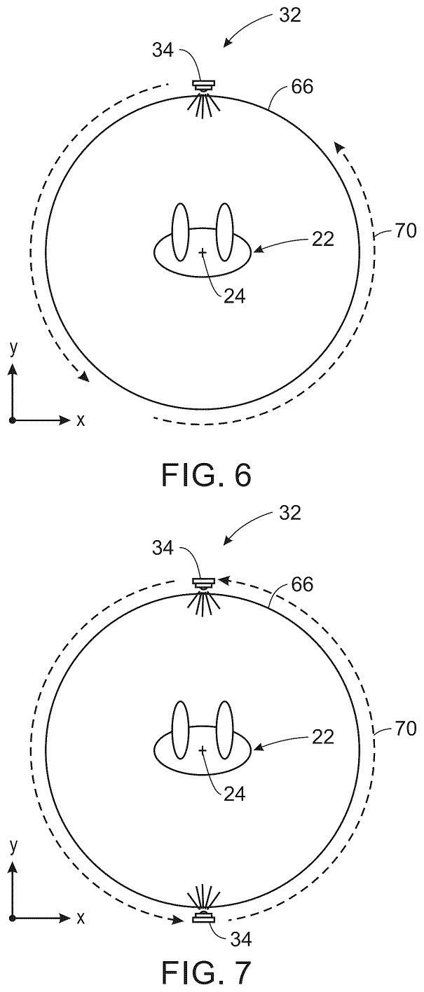

FIGS. 5A-5C depict a single motion detection apparatus 34 obtaining data at different angular positions. With the exception of the motion detection system 32, the components of the gantry are not shown for simplification. As depicted in FIGS. 5A-5C, the single motion detection apparatus 34 is coupled to the panel 66. The motion detection apparatus 34 rotates (e.g., about the axis of rotation 24 in FIG. 2) to acquire the movement data. In particular, the motion detection system 34 (and panel 66) rotates to different angular positions to acquire different views of movement data. The data acquired at the different angular positions can then be combined later to generate the 3D measurement of the patient 22. It should be noted although the patient 22 is depicted outside an area of the gantry where the panel 66 is located, in certain embodiments, the patient 22 may be moved into the gantry via the table 36 so that the panel 66 is disposed about a portion of the patient 22. As depicted in FIG. 6, the single motion detection apparatus 34 may rotate up to 360 degrees in a circumferential direction 70 (e.g., relative to the axis of rotation 24) around the target (i.e., the patient 22) to acquire the movement data at the different angular positions.

In certain embodiments, as depicted in FIG. 7, multiple motion detection apparatuses 34 may be coupled to the panel 66. As depicted, two motion detection apparatuses 34 are coupled to the panel 66. The number of motion detection apparatuses 34 coupled to the panel 66 may vary (e.g., 2, 3, 4, 5, 6, or other number of motion detection apparatuses 34). The motion detection apparatuses 34 are circumferentially 70 spaced apart from each other along the panel 66. Each motion detection apparatus 34 (and the panel 66) rotates about the axis of rotation 24. In certain embodiments, each motion detection apparatus 34 rotates only a portion of the 360 degrees around the patient 22 to acquire the movement data. For example, as depicted in FIG. 7, each motion detection apparatus 34 may rotate only up to approximately 180 degrees circumferentially 70. Thus, together both the motion detection apparatuses 34 may cover 360 degrees about the patient 22. In certain embodiments, with three motion detection apparatuses 34 coupled to the panel 66, each motion detection apparatus 34 may cover up to approximately 120 degrees circumferentially 70. In certain embodiments, with four motion detection apparatuses 34 coupled to the panel 66, each motion detection apparatus 34 may cover up to approximately 90 degrees circumferentially 70. Thus, no matter the number of motion detection apparatuses 34 coupled to the panel 66, each motion detection apparatus 34 may cover an equal portion of the 360 degrees about the patient 22. In certain embodiments, when multiple motion detection apparatus 34 are coupled to the panel 66, one or more of the motion detection apparatuses 34 cover a different angular range about the patient 22 relative to the other motion detection apparatuses 34. For example, in the case of two motion detection apparatuses 34, one motion detection apparatus 34 may cover 120 degrees and the other motion detection apparatus may cover 240 degrees. In certain embodiments, when multiple motion detection apparatuses 34 are coupled to the panel 66, each motion detection apparatus 34 may cover up to 360 degrees to provide redundancy in the acquired data. In certain embodiments, the motion detection system 32 may not cover 360 degrees about the patient 22 when certain angular positions are obstructed (e.g., by a bottom of the CT table).

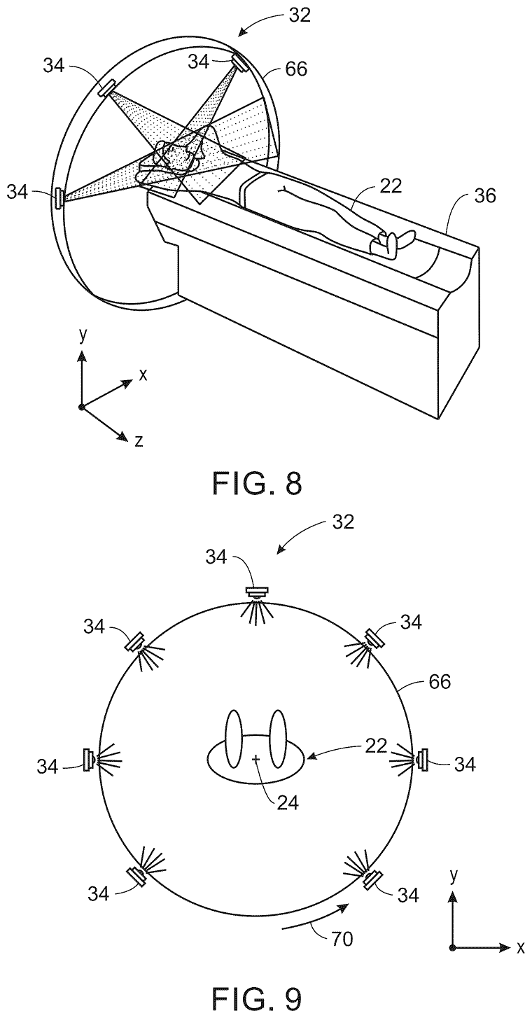

FIGS. 8 and 9 illustrate utilizing multiple motion detection apparatuses 34 to obtain data of the patient 22 at different angular positions at the same time. With the exception of the motion detection system 32, the components of the gantry are not shown for simplification. As depicted, the motion detection system 32 includes multiple motion detection apparatuses 34 coupled to the panel 66. The motion detection apparatuses 34 are circumferentially 70 spaced apart (e.g., relative to the axis of rotation 24) from each other along the panel 66. The motion detection apparatuses 34 are disposed in fixed positions (e.g., different angular positions) along the panel 66. Both the motion detection apparatuses 34 and panel 66 remain stationary during acquisition of the movement data. In particular, movement data is acquired by each of the motion detection apparatuses 34 scanners at the same time, thus, providing movement data at different views (i.e., different angular positions). The data acquired at the different angular positions can then be combined later to generate the 3D measurement of the patient 22.

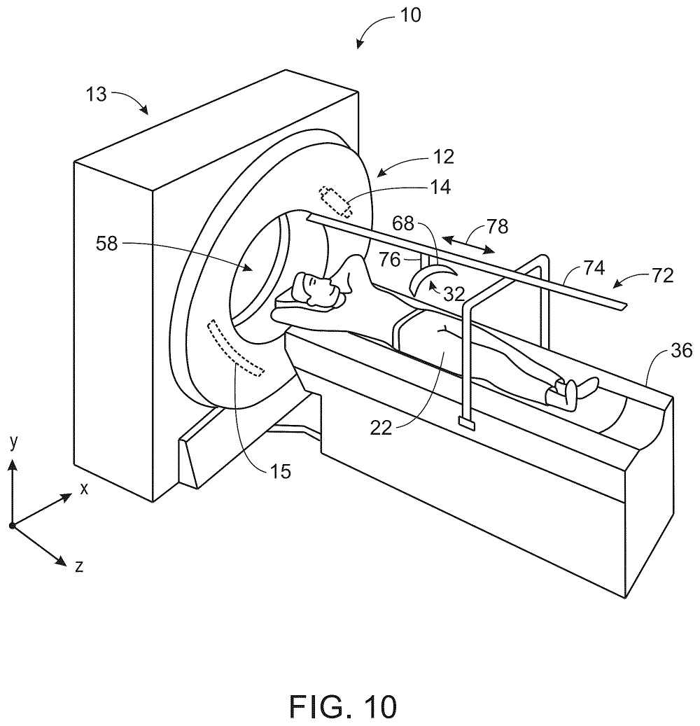

In certain embodiments, the motion detection system 32 may be external to the gantry 12 but still physically coupled to the imaging system 10. FIG. 10 is a pictorial representation of an external motion detection system 32 coupled to the CT table 36. The motion detection system 32 is coupled to a guide rail system 72. The guide rail system 72 is coupled to the table 36. The guide rail system 72 is configured to move the motion detection system 32 toward and away from the gantry 12 (e.g., in the Z-direction along the longitudinal length of the patient 22). The guide rail system 72 includes a main guide rail 74 coupled to a support structure 76 that supports the main guide rail 74. The main guide rail 74 extends in the Z-direction above the table 36 (e.g., along a longitudinal length of the table 36). The support structure 76 is coupled to the table 36. The support structure 76 extends in a vertical direction (e.g., in the Y-direction) from the table 36 and then extends in a horizontal direction (e.g., in the X-direction) to couple to the main guide rail 74. The configuration of the support structure 76 and how it is coupled to both the table 36 and the main guide rail 74 may vary.

The motion detection system 32 includes the panel 68 (e.g., having an arc shape). The panel 68 includes a plurality of motion detection apparatuses coupled to it. The plurality motion detection apparatus are circumferentially spaced apart along the arc of the panel 68 at different angular positions to enable the acquisition of different views of movement data. The panel 68 is coupled to the guide rail system 72. In particular, the panel 68 is coupled to the main guide rail 74 via a vertical support stanchion 76 (e.g., post or bar). The guide rail system 72 is configured to move the panel 68 (and the vertical support stanchion) back and forth toward the gantry 12 as indicated by arrow 78 (in the Z-direction) along the main guide rail 74. The main guide rail 74 may include an actuation system (e.g., chain actuator, electro-mechanical linear actuator, or any other mechanism for facilitating linear movement along the main guide rail 74). In certain embodiments, the guide rail system 72 is configured to circumferentially move the panel 68 along its arc. In particular, the guide rail system 72 may enable the panel 68 to rotate circumferentially (e.g., relative to the axis of rotation 24 in FIG. 2) so that the panel 68 is moving with respect to the vertical support stanchion 76. The panel 68 may rotate both clockwise and counter-clockwise (e.g., relative to the axis of rotation 24 in FIG. 2). Rotational movement of the panel 68 enables the acquisition of movement data from additional different angular positions.

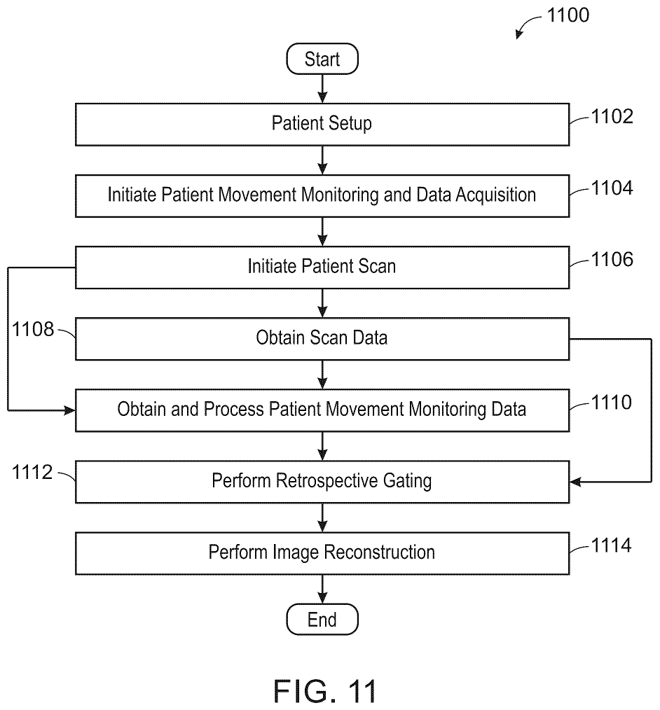

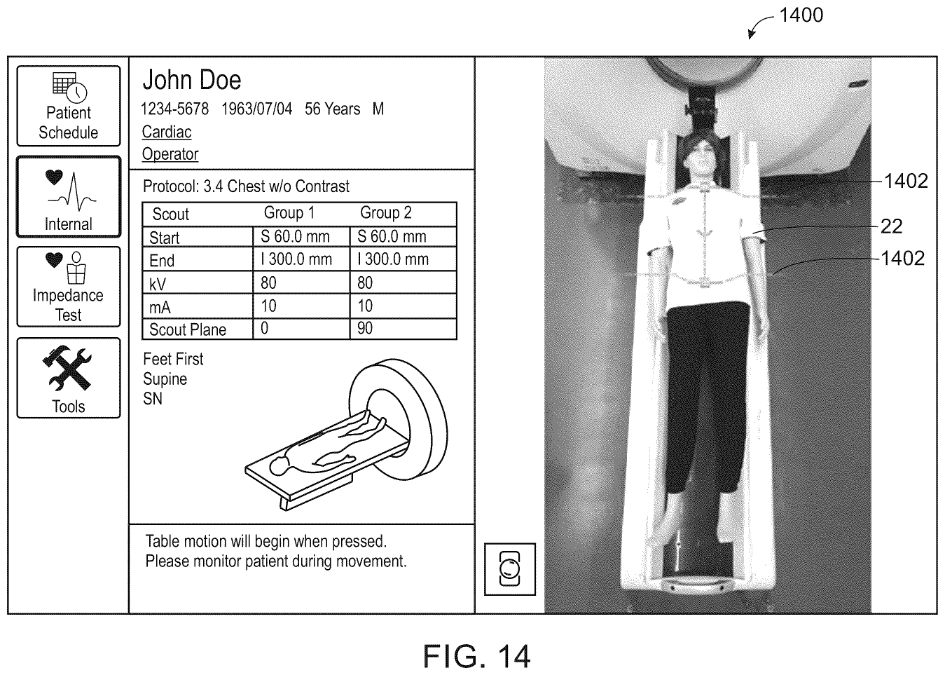

FIG. 11 depicts a flowchart of a method 1100 for retrospective gating based on motion detected during scan data acquisition. The method begins at block 1102 with the patient setup, including the being positioned on the table 36 of the CT imaging system 10 and an initial input or conformation of patient data in a graphical user interface of the CT system (as depicted in FIG. 14). In some examples, a user or technician assists with the patient placement on the table. In other examples, a patient may position themselves on the table with assistance from the technician and/or imaging system. Patient setup may also include the CT imaging system obtaining the scan protocol. For example, the computer 42 associated with the CT imaging system obtains the scan protocol from mass storage 50, a remote server, and/or patient information. At block 1104, patient movement monitoring and data acquisition begins. Patient movement monitoring and data acquisition may include the use of any suitable motion detection system, including but not limited to LiDAR systems, 3D cameras, and other motion detectors. The monitoring and data acquisition is done continuously and in real-time for the duration of the scan.

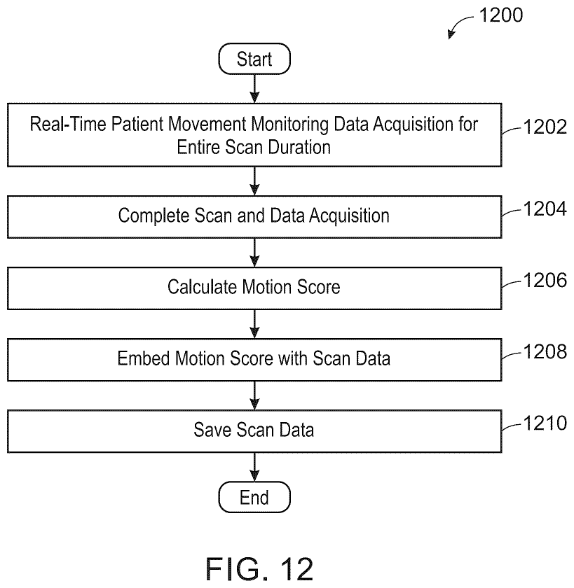

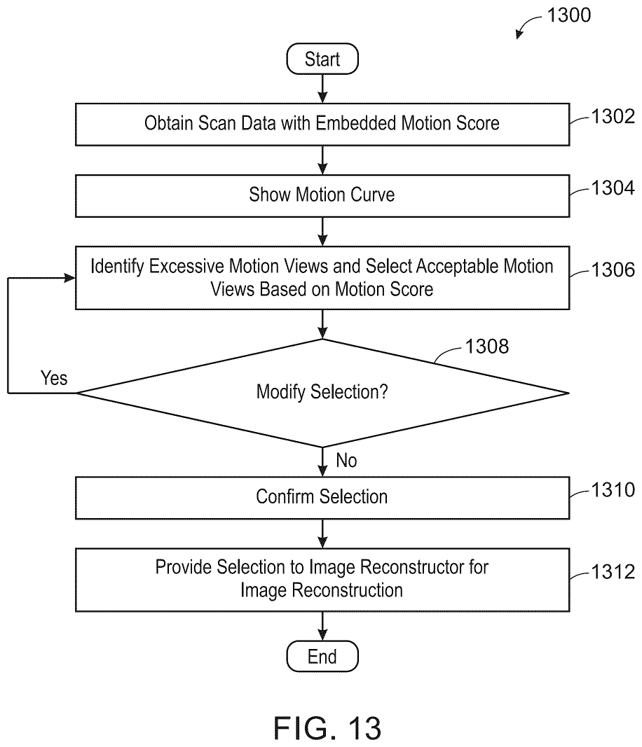

At block 1106, the scan is initiated and patient scan data acquisition begins. During the patient scan, scan data is obtained simultaneously with movement data (blocks 1108 and 1110). Additionally, the movement data may be processed in real-time to determine a motion score. More details of an example method for processing patient movement monitoring data are described in conjunction with FIG. 12. In block 1112, the data is used to perform retrospective gating, which removes portions of the scan data that contain movement above a threshold. The method described in conjunction with FIG. 13 provides more details on the retrospective gating. After retrospective gating is complete, the method 1100 continues with performing image reconstruction with the remaining scan data. Image reconstruction may include any known methods of reconstruction suitable for use with CT scan data. After reconstruction, the method 1100 is complete.

FIG. 12 depicts a flowchart of a method 1200 for determining a motion score based on the movement data. The method 1200 begins with real-time patient movement monitoring data acquisition to collect movement data of the patient. The movement data is collected for the entire duration of the CT scan (block 1202). When the scan and data acquisition is completed (block 1204), a motion score is calculated (block 1206). The motion score is a quantitative metric that may be used to determine whether the patient's motion during the scan exceeds a threshold. The motion score is then embedded with the scan data (block 1206). That is, the motion score at each point in time of the scan is calculated and synchronized with the scan data so that the motion score can be identified at each point of the scan. The scan data with the embedded motion score is saved for later user (block 1210) and the method 1200 is complete.

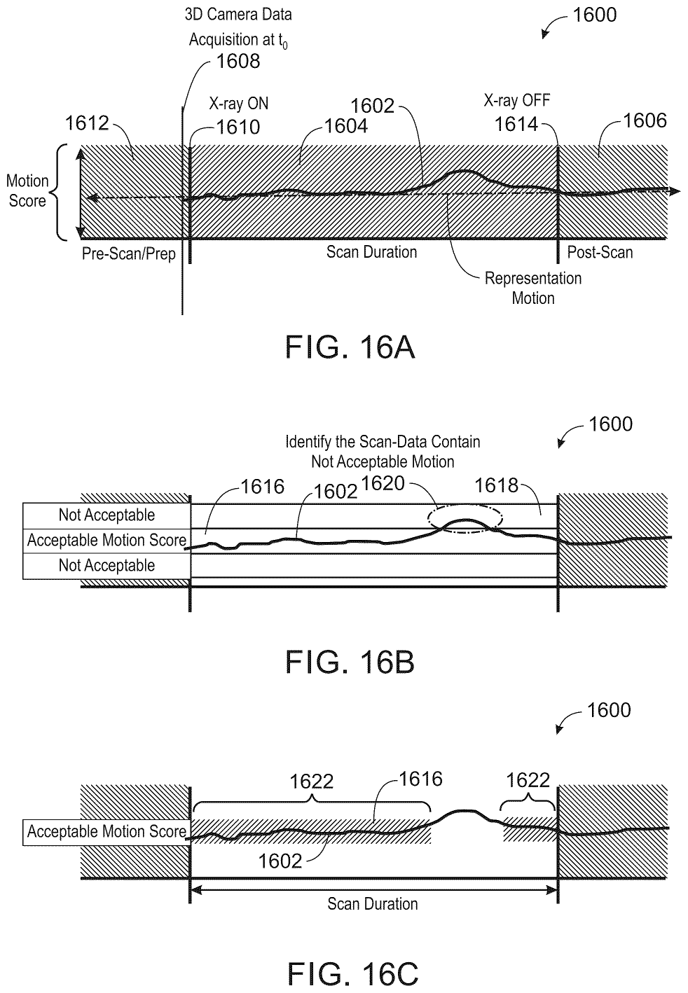

FIG. 13 depicts a flowchart of a method 1300 for retrospective gating of views of a scan that include excessive motion. The method 1300 begins by obtaining the scan data with embedded motion scores (block 1302). FIG. 16A depicts an example set of scan data embedded with a motion score. The example motion score can be depicted in the form of a motion score curve, indicating the amount of motion at each point in time during the scan (block 1304). The example motion score curve is presented to a user or technician via a graphical user interface. From the motion curve, the views with excessive motion are identified based on the motion score associated with each view. For example, any point on the curve depicting that the motion score is outside of an acceptable motion score is identified and the views with acceptable motion are selected (block 1306). In some examples, the views with excessive motion are identified and the remaining views are initially selected automatically (e.g., by the data correction unit) for use in reconstruction. Alternatively, the views can be selected manually by a user or technician. If the selection of views with acceptable motion should be modified (block 1308), the user may manually identify and/or removing the views with excessive motion and select the views with acceptable motion. The user may change the selection using the graphical user interface. If the selection does not need to be modified, the method continues to block 1310, where the selection is confirmed by the user or technician. The selection is then provided to the image reconstructor for image reconstruction 1312. The method 1300 is complete.

FIG. 14 depicts an example user interface 1400 of patient setup, in which the chest of a patient 22 is to be scanned. The surface area of interest is represented by the dashed lines 1402. These lines 1402 can be adjusted based on the size of the patient 22, and can be used to ensure that the correct area of interest is scanned. Additionally, the patient information and scan protocol information are displayed so that the user can confirm the information or adjust the information if needed. The scan may also be initiated from the user interface.

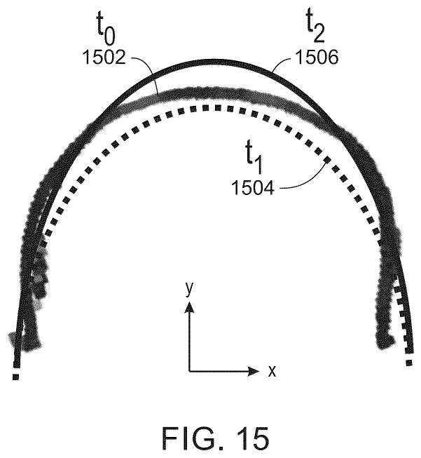

FIG. 15 depicts an example representation of determining a motion score for a contour of a patient. The initial or neutral state 1502 of the patient may be represented by t₀. Different amounts of motion are represented by the contours 1504 and 1506 (i.e., t₁ and t₂). For example, the motion t₁ and t₂ may be the result of the chest moving due to the patient breathing, or may be the result of a sudden, larger movement of the patient. Specifically, the motion that may fall outside of the acceptable range may be the result of unexpected patient movement, rather than the repeatable, cyclical motion of the patient breathing. The motion score of each movement can be calculated to determine whether the movement falls out of an acceptable range of movement. For a contour 1502, 1504, 1506 of a patient, the motion score can be calculated using Equation 1:

$$motion {score}_{{t}_{n}} = \sqrt{{( {x}_{{t}_{n}} - {x}_{{t}_{0}} )}^{2} + {( {y}_{{t}_{n}} - {y}_{{t}_{0}} )}^{2}} \qquad Equation 1$$

In Equation 1, the motion score can be determined for each contour at a point in time during the scan. The position of the contour tₙ is compared to the initial or baseline position to, where n represents the number of the point in time or the contour 1504, 1506 being evaluated. The position of the contour 1504, 1506 in both x and y directions are used to determine the overall motion score. The motion score may also be given a direction in addition to a magnitude. For example, the motion score for contour 1504 might be negative because the movement is a reduction in the y-direction from the initial contour 1502, and the motion score for 1506 might be positive because the movement is an increase in the y-direction from initial contour 1502. The position of the patient may be determined based on the point cloud or data mesh created using the movement data acquired using the motion detection apparatuses. For example, if one or more of the motion detection apparatuses includes a LiDAR scanner, a 3D point cloud may be created to identify the position of the patient at any given time during the scan, and the real-time position or movement data of the patient can be compared to the initial position or initial point cloud information of the patient to determine the motion score. Similarly, if the motion detection apparatuses include a 3D camera, the 3D mesh created by the camera may be compared in real-time to an initial or baseline 3D mesh to determine the motion score. Thus, the motion score for each view is determined based on the initial position information and the real-time or additional position information obtained for the duration of the scan.

For the surface of a patient, the motion score can be calculated using Equation 2:

$$motion {score}_{{t}_{n}} = \sqrt{{( {x}_{{t}_{n}} - {x}_{{t}_{0}} )}^{2} + {( {y}_{{t}_{n}} - {y}_{{t}_{0}} )}^{2} + {( {z}_{{t}_{n}} - {z}_{{t}_{0}} )}^{2}} \qquad Equation 2$$

To obtain the motion score for the surface of the patient, Equation 2 also incorporates a z-direction component. The motion scores may be calculated for the entire surface of interest for the patient at each point in time for the duration of the scan. The motion scores are then embedded in the scan data and provided to the user in the graphical representation of a curve indicating which views have a motion score that exceeds an acceptable threshold.

FIGS. 16A-16C depict the example graphical representation of the curve 1602 indicating the motion scores throughout the scan. As shown in FIG. 16A, the motion score fluctuates for the duration of the scan 1604 and post-scan 1606 areas of the graph. The motion data acquisition begins at a time 1608 just prior to the scan data acquisition start time 1610, during a pre-scan 1612 or preparation area of the graph. While this example depicts the motion data acquisition continuing after the scan is complete 1614, the motion data acquisition may also end when the scan data acquisition ends.

FIG. 16B depicts an example graph including an acceptable motion score range 1616 and a range for a motion score that is not acceptable 1618. For example, when the motion score exceeds a threshold, the motion score enters the not acceptable range. As depicted in FIG. 16B, a portion 1620 of the scan views correspond to a motion score that exceeds the threshold and falls within a not acceptable range 1618. This portion 1620 is identified as not acceptable and the remaining portion(s) or acceptable portion(s) 1622 of the scan views are selected for use in the image reconstruction, as depicted in FIG. 16C. For some scans, such as axial scans, the remaining portions of the scan data must meet a threshold length to be sufficient for reconstruction. For example, the continuous length of an acceptable portion of data must have a length that is at least two-thirds of the duration of the scan.

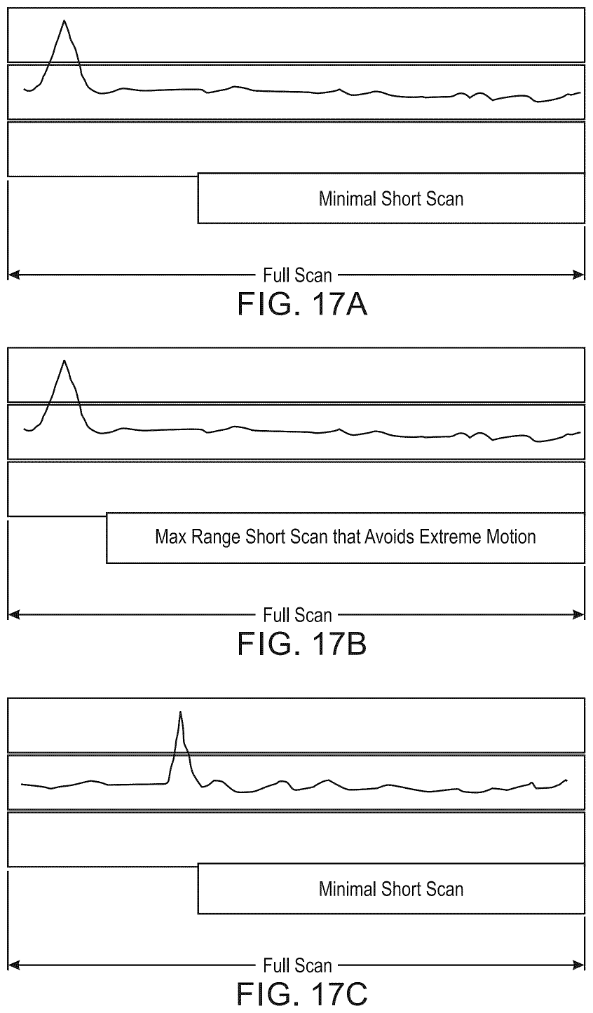

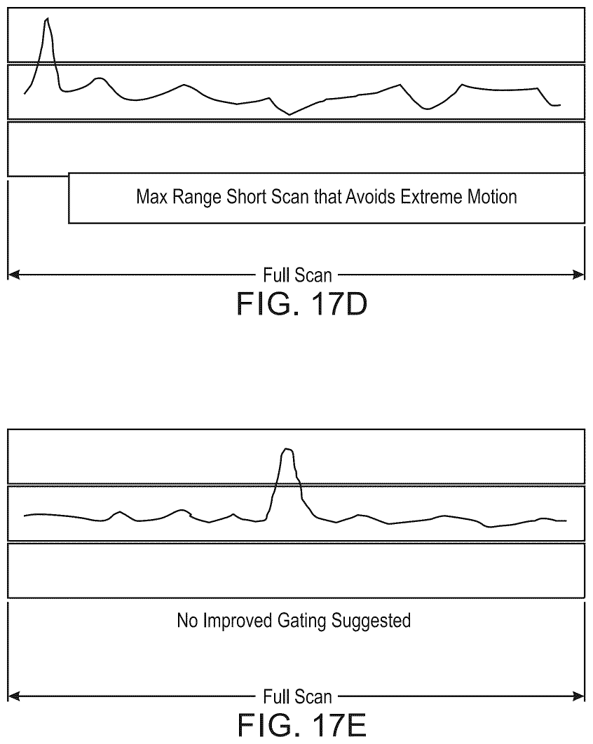

FIGS. 17A-17E depict additional samples of motion data during a scan. FIGS. 17A and 17B depict an example set of motion data that includes a portion of views that has a motion score exceeding an acceptable range near the beginning of the scan. This set of data is acceptable for reconstruction because the acceptable scan data has a length that is longer than a minimal short scan (FIG. 17A) and has a maximum range of the short scan that avoids extreme motion that is greater than two-thirds of the duration of the scan. FIG. 17C also depicts a set of data that is acceptable for reconstruction because the acceptable data has a continuous length that is longer than a minimal short scan (e.g., two-thirds of the duration of the total scan). FIG. 17D depicts scan data that includes more motion overall (e.g., the motion score fluctuates more than the previously depicted sets of scan data), but much of the motion is still in the acceptable range and the acceptable portion of the data is sufficient for reconstruction.

FIG. 17E depicts an example set of data that is not sufficient for construction. In FIG. 17E, the portion of the data corresponding to a motion score outside of the acceptable range is in the middle of the scan duration. Thus, neither section of the scan data corresponding to an acceptable motion score is sufficient in length for reconstruction. After seeing the motion score for this set of data, the user or technician will re-start the scan to obtain a data set that is acceptable for the reconstruction.

While the example methods are described as a whole, portions of the methods may be used independently of the remainder of the methods. In other examples, other portions of the methods 1100, 1200, and 1300 may be excluded, or additional steps may be included and/or repeated. Although a CT system is described by way of example, it should be understood that the present techniques may also be useful when applied to images acquired using other imaging modalities, such as X-ray imaging systems, magnetic resonance (MR) imaging systems, positron emission tomography (PET) imaging systems, single-photon emission computed tomography (SPECT) imaging systems, ultrasound imaging systems, and combinations thereof (e.g., multi-modality imaging systems, such as PET/CT, PET/MR or SPECT/CT imaging systems). The present discussion of a CT imaging modality is provided merely as an example of one suitable imaging modality.

Technical effects of the disclosed embodiments include providing systems and methods for generating an accurate representation of movement of a patient during a CT scan to be utilized in a subsequent CT workflow process for the CT scan. The present disclosure provides systems and methods for incorporating motion detection techniques, which may include LiDAR based techniques, with a CT imaging system to aid various workflows more efficiently. The disclosed embodiments provide a method for detecting the movement of a subject during an imaging scan and subsequently select views of the scan containing an acceptable amount of movement based on the detected movement, thereby increasing the image quality.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims. Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

1. A method of identifying movement of a patient during a medical imaging scan, the method comprising:
initiating motion detection data acquisition;
initiating a medical imaging scan of the patient to acquire scan data;
determining a motion score curve for duration of the medical imaging scan based on the motion detection data;
removing portions of the scan data corresponding to a motion score curve outside of an acceptable range; and
selecting the scan data corresponding to a motion score curve within the acceptable range for reconstruction.

2. The method of claim 1, wherein the motion score curve is a compilation of motion scores calculated for each view for the duration of the scan.

3. The method of claim 2, further comprising determining a baseline position of the patient used to determine the motion score for each view.

4. The method of claim 1, wherein the motion score curve is determined for a contour of the patient.

5. The method of claim 1, wherein the motion score curve is determined for a surface of the patient.

6. The method of claim 1, wherein selecting the scan data corresponding to a motion score curve within the acceptable range is automatically determined by a data correction unit and wherein a user confirms the selection.

7. The method of claim 1, wherein the motion detection data acquisition uses one or more motion detection apparatuses to detect motion in real-time during the scan.

8. The method of claim 7, wherein the motion detection apparatuses include a LiDAR scanner.

9. The method of claim 7, wherein the motion detection apparatuses include a 3D camera.

10. A medical imaging system, comprising:
a computed tomography (CT) imaging system, comprising:
a gantry having a bore, rotatable about an axis of rotation;
an X-ray source mounted on the gantry and configured to emit an X-ray beam;
an X-ray controller to operate the X-ray source; and
an X-ray detector configured to detect the X-ray beam emitted by the X-ray source;
a motion detection system coupled to the CT imaging system, wherein the motion detection system includes:
a motion detection apparatus mounted on the gantry;
a controller to control operation of the motion detection apparatus; and
a movement data processing unit to obtain data from the motion detection apparatus, wherein the movement data processing unit obtains a baseline position of a patient and real-time movement data during an imaging scan to generate additional position information, wherein the real-time movement data is compared to the baseline position of the patient; and
a processor to determine a motion score of the patient for each view during the scan and select views of scan data in which the corresponding motion score is in an acceptable range for image reconstruction.

11. The system of claim 10, further comprising determining a motion score curve from a compilation of the motion scores calculated for each view for the duration of the scan.

12. The system of claim 10, wherein the motion score is based on the baseline position of the patient and the real-time movement data corresponding to each view during the scan.

13. The system of claim 10, wherein the motion score is determined for a contour of the patient.

14. The system of claim 10, wherein the motion score is determined for a surface of the patient.

15. The system of claim 10, wherein the processor includes a data correction unit to automatically select the views of the scan data corresponding to the motion score within the acceptable range.

16. The system of claim 10, wherein the motion detection apparatus includes a LiDAR scanner.

17. The system of claim 10, wherein the motion detection apparatus includes a 3D camera.
